(19) United States
(12) Patent Application Publication
(10) Pub. No.: US 2011/0087108 A1
(43) Pub. Date: Apr. 14, 2011

(54) SELF-LUMINOUS SENSOR DEVICE

(75) Inventors: Atsushi Onoe, Saitama (JP); Yoshinori Kimura, Saitama (JP)

(73) Assignee: PIONEER CORPORATION, Kawasaki-shi, Kanagawa (JP)

(21) Appl. No.: 12/991,968

(22) PCT Filed: May 12, 2008
(86) PCT No.: PCT/JP2008/058695

§ 371 Date: Dec. 13, 2010

(51) Int. Cl. A61B 6/00

(52) U.S. Cl. 600/473

(57) ABSTRACT

A light-emitting sensor device includes: a substrate (110); and disposed thereon an irradiating part (120) for applying light to a specimen; a light receiving part (160) for detecting light from the specimen caused by the applied light; and a cap, which has (i) a main body for accommodating at least one of the irradiating part and the light receiving part and (ii) a reflective light shielding film (252) which is one portion of a surface of the main body, which is formed on an inclined surface inclined to a surface of the substrate, which reflects the light emitted from the irradiating part to go to the specimen, and which blocks incidence of the light emitted from the irradiating part to the light receiving part. The light-emitting sensor device is suitable for mass production, and enables highly accurate detection of a predetermined type of information on a specimen.

### TECHNICAL FIELD

The present invention relates to a light-emitting sensor device capable of measuring a blood flow velocity or the like.

### BACKGROUND ART

As this type of light-emitting sensor device, there is a device for applying light such as laser light to a living body and for calculating the blood flow velocity of the living body from a change in wavelength by Doppler shift in its reflection or scattering (e.g. refer to patent documents 1 to 4). In this type of light-emitting sensor device, typically, miniaturization is expected by providing a light source such as a semiconductor laser for applying light to a living body and a light detector such as a photodiode for detecting light from the living body to be close to each other, in an enclosure or housing. Moreover, in most cases, such a light-emitting sensor device has a light shielding structure for preventing light which should not be detected, such as light directly going to the light detector without being applied to the living body, out of light from the light source, from being detected by the light detector. Moreover, if an edge-emitting semiconductor laser is used as the light source, a light reflecting device for defining the optical path of the light from the semiconductor laser is provided in most cases.

For example, in a patent document 1, the aforementioned light shielding structure is realized by providing a light shielding plate between the semiconductor laser and the photodiode in the enclosure. At the same time, as the aforementioned light reflecting device, a reflection plate is provided which makes about 45 degrees with respect to the irradiation direction of the laser light from the light source. In a patent document 2, the aforementioned light shielding structure is realized by separately disposing the semiconductor laser and the photodiode in each of two concave portions formed by performing an anisotropy etching process on a silicon substrate. At the same time, a metal film for mirror is formed as the aforementioned light reflecting device on the inner surface of the concave portion.
Patent document 1: Japanese Patent Application Laid Open No. 2004-357784
Patent document 2: Japanese Patent Application Laid Open No. 2004-229920
Patent document 3: Japanese Patent Application Laid Open No. 2002-330936
Patent document 4: Japanese Patent Application Laid Open No. 2006-130208

### DISCLOSURE OF INVENTION

### Subject to be Solved by the Invention

However, according to the technologies disclosed in the patent documents 1 and 2 described above, there is such a technical problem that processes requiring a lot of time increase and the number of the processes increases in a manufacturing process of manufacturing the light-emitting sensor device. Thus, a yield in the manufacturing process likely decreases, resulting in an increase in manufacturing cost of the device.

For example, in the technology disclosed in the patent document 1, it is necessary to incorporate relatively many parts in the enclosure including the aforementioned light shielding plate, reflective plate, or the like in addition to the semiconductor laser and the photodiode. Thus, the number of processes likely increases, and it likely requires a lot of time for the positioning of the parts.

Moreover, in the technology disclosed in the patent document 2, for example, a small sensor device which is several millimetersxseveral millimeters in size can be realized; however, it likely takes a lot of time to perform the anisotropy etching process for forming the concave portion on the silicon substrate, and the yield likely decreases due to variations in the manufacture caused by the anisotropy etching process. Moreover, since the concave portion is formed on the silicon substrate by the anisotropy etching process, the inclination angle of an inclined surface of the concave portion in which the metal film for mirror is formed is limited to almost a certain angle, such as 54.7 degrees, depending on the crystal structure of silicon.

In view of the aforementioned problems, it is therefore an object of the present invention to provide a small light-emitting sensor device, which is suitable for mass production and which can detect a predetermined type of information such as a blood flow velocity on a specimen, highly accurately.

### Means for Solving the Subject

The above object of the present invention can be achieved by a light-emitting sensor device provided with: a substrate; an irradiating part, disposed on the substrate, for applying light to a specimen; a light receiving part, disposed on the substrate, for detecting light from the specimen caused by the applied light; and a cap, disposed on the substrate, which has (i) a cap main body for accommodating at least one of the irradiating part and the light receiving part and (ii) a reflective light shielding film which is one portion of a surface of the cap main body, which is formed on an inclined surface inclined to a substrate surface of the substrate, which reflects the light emitted from the irradiating part to go to the specimen, and which blocks incidence of the light emitted from the irradiating part to the light receiving part.

According to the light-emitting sensor device of the present invention, in its detection, the light such as laser light is applied to the specimen, which is one portion of a living body, by the irradiating part including e.g. an edge-emitting semiconductor laser. Here, the light emitted from the irradiating part typically along the substrate surface of the substrate is reflected by the reflective light shielding film and thus goes to the specimen. The light from the specimen caused by the light applied to the specimen in this manner is detected by the light receiving part including e.g. a light receiving element. Here, the “light from the specimen caused by the light applied to the specimen” means light caused by the light applied to the specimen, such as lights reflected, scattered, diffracted, refracted, transmitted through, Doppler-shifted in the specimen and interfering light by the above lights. On the basis of the light detected by the light receiving part, it is possible to obtain predetermined information such as a blood flow velocity associated with the specimen.

Particularly in the present invention, the cap is provided, which has the cap main body made of a resin or the like and the reflective light shielding film formed on one portion of the surface of the cap main body. The reflective light shielding film reflects the light emitted from the irradiating part to go to the specimen. Thus, it is possible to make it certain that the light emitted from the irradiating part emits the specimen. Moreover, the reflective light shielding film blocks the incidence of the light emitted from the irradiating part to the light receiving part; namely, the reflective light shielding film blocks the light directly going from the irradiating part to the light receiving part. In other words, the light which is emitted from the irradiating part and which goes to the light receiving part without being applied to the specimen is; blocked by the reflective light shielding film. Therefore, it is possible to prevent that the light detected by the light receiving part changes due to the light directly going from the irradiating part to the light receiving part. As a result, it is possible to detect a predetermined type of information, such as a blood flow velocity, on the specimen, highly accurately.

Moreover, particularly in the present invention, the reflective light shielding film is formed on the inclined surface which is one portion of the surface of the cap main body made of a resin or the like, so that it is possible to simplify or reduce each process in a manufacturing process. By this, it is possible to increase a yield and to reduce manufacturing cost as well. In addition, for example, by forming the cap main body of a resin, glass, or the like, it is possible to arbitrarily set the inclination angle of the inclined surface on which the reflective light shielding film is to be formed. In other words, in comparison with a case where the inclined surface is formed by performing an anisotropy etching process on a silicon substrate, the inclination angle of the inclined surface can be arbitrarily selected.

As explained above, according to the light-emitting sensor device of the present invention, it is possible to detect the predetermined type of information, such as a blood flow velocity, on the specimen, highly accurately. Moreover, it is possible to increase the yield and to reduce the manufacturing cost, and it is suitable for mass production.

In one aspect of the light-emitting sensor device of the present invention, the cap main body is formed of a resin, and a light shielding film is formed at least partially on a surface other than the inclined surface out of a surface of the cap main body.

According to this aspect, it is possible to increase the processability of the cap main body. Moreover, by virtue of the light shielding film, it is possible to reduce that unnecessary light from the surroundings of the light-emitting sensor device enters the irradiating part or the light receiving part.

In another aspect of the light-emitting sensor device of the present invention, the cap main body accommodates the light receiving part as the at least one and has a pore for transmitting light from the specimen.

According to this aspect, of the irradiating part and the light receiving part, only the light receiving part is accommodated within the cap. In the detection, the light from the specimen enters the light receiving part via the pore (i.e. pinhole). By the pore, the light entering the light receiving part is limited. Thus, it is possible to prevent light which does not have to be detected from entering the light receiving part, thereby increasing detection accuracy. Incidentally, a transparent member may be formed in a part or all of the inside of the pore.

In another aspect of the light-emitting sensor device of the present invention, the irradiating part has a plurality of light sources, and the cap main body has a plurality of inclined surfaces, each of which is formed in accordance with respective one of a plurality of lights emitted from the plurality of light sources and which are inclined to the substrate surface at mutually different angles.

According to this aspect, the lights emitted from the plurality of light sources, which are a plurality of edge-emitting semiconductor lasers, can be reflected by the reflective light shielding film formed on the plurality of inclined surfaces which are inclined at the mutually different angles, to mutually different portions on the specimen. Thus, it is possible to detect the predetermined information, such as a blood flow velocity, in the plurality of mutually different portions on the specimen, more quickly. In other words, it is possible to detect the predetermined information, such as a blood flow velocity, in the plurality of portions on the specimen, without changing a relative position relation between the specimen and the light-emitting sensor device,

In an aspect in which the cap main body has the plurality of inclined surfaces, as described above, the plurality of light sources may be a plurality of semiconductor lasers, each of which emits respective one of laser lights with mutually different wavelengths.

In this case, the laser light has such a character that it has a different penetration force to a living body or the like depending on a difference in wavelength. By using such a character, it is possible to perform the measurement in different depths of the specimen.

In an aspect in which the plurality of light sources are the plurality of semiconductor lasers, each of which emits respective one of laser lights with mutually different wavelengths, as described above, the plurality of inclined surfaces are arranged such that a plurality of reflected lights, obtained by reflecting the plurality of lights with the reflective light shielding film, are applied to a same portion on the specimen.

In this case, for example, it is possible to detect the predetermined information such as a blood flow velocity, by applying the laser lights with the mutually different wavelengths to the same portion on the specimen. Thus, it is also possible to further increase the accuracy of the detection of the predetermined information such as a blood flow velocity. Incidentally, the expression that “the reflected lights are applied to the same portion on the specimen” means that the reflected lights are applied with them at least partially overlapping with each other with respect to the specimen, and the “same portion” can mean a portion with mutually different depths in terms of the depth direction of the specimen.

In another aspect of the light-emitting sensor device of the present invention, the cap main body accommodates the irradiating part as the at least one and is made of a transparent member which can transmit the light emitted from the irradiating part, the inclined surface is one portion of an outer surface located on a side which is not opposed to the irradiating part, out of a surface of the cap main body, and the cap main body has a refracting surface which refracts the light emitted from the irradiating part to go to the reflective light shielding film.

According to this aspect, the light emitted from the irradiating part is refracted by the refracting surface, is transmitted through the inside of the cap main body, and then is reflected by the reflective light shielding film formed on the inclined surface, which is one portion of the outer surface of the cap main body, to go to the specimen. Thus, for example, by changing the inclination angle of each of the refracting surface and the inclined surface to the substrate surface, it is possible to change the path of the light emitted from the laser diode to the specimen. In other words, in designing the path of the light emitted from the laser diode to the specimen, the inclination angles of the refracting surface in addition to the inclined surface can be set as design parameters (i.e. the degree of freedom of designing can be increased).

In another aspect of the light-emitting sensor device of the present invention, the cap main body accommodates the irradiating part as the at least one and is made of a transparent member which can transmit the light emitted from the irradiating part, the inclined surface is one portion of an outer surface located on a side which is not opposed to the irradiating part, out of a surface of the cap main body, and the light-emitting sensor device further comprises a resin part formed of a light shielding resin to cover the reflective light shielding film and to surround the light receiving part.

According to this aspect, by virtue of the resin part, it is possible to prevent the oxidation of the reflective light shielding film made of a metal reflective film, such as a silver film and an aluminum film, and it is possible to reduce that the unnecessary light from the surroundings of the light receiving part enters the light receiving part.

In another aspect of the light-emitting sensor device of the present invention, it is further provided with a light receiving part upper surface light shielding film, which is disposed on an upper surface of the light receiving part, which is made of a light shielding material, and which is to transmit light from the specimen.

According to this aspect, the upper surface of the light receiving part is covered by the light receiving part upper surface light shielding film. In the detection, the light from the specimen enters the light receiving part via the pore. The light entering the light receiving part is limited by the pore. Thus, it is possible to prevent the light which does not have to be detected from entering the light receiving part, thereby increasing the detection accuracy.

In another aspect of the light-emitting sensor device of the present invention, the cap main body accommodates the irradiating part and the light receiving part and is made of a transparent member which can transmit the light emitted from the irradiating part, the inclined surface is one portion of a light-receiving-part-side inner surface opposed to the light receiving part, out of a surface of the cap main body, and one portion of an irradiating-part-side inner surface opposed to the irradiating part out of the surface of the cap main body is formed as a refracting surface which refracts the light emitted from the irradiating part to go to the reflective light shielding film.

According to this aspect, the irradiating part and the light receiving part can be protected by the cap main body. Thus, the durability or reliability of the light-emitting sensor device can be increased.

In another aspect of the light-emitting sensor device of the present invention, the irradiating part has an edge-emitting semiconductor laser for emitting laser light along the substrate surface as the light.

According to this aspect, the laser light can be applied by applying a voltage to the semiconductor of the irradiating part such that an electric current flows with a higher value than a laser oscillation threshold value. The laser light has such a character that it has a different penetration force to a living body or the like depending on a difference in wavelength. By using such a character, it is possible to perform the measurement in various depths of the specimen.

Moreover, the irradiating part has an edge-emitting semiconductor laser such as a Fabry-Perot (FP) laser which is relatively inexpensive, so that it is possible to further reduce the manufacturing cost.

In another aspect of the light-emitting sensor device of the present invention, it is further provided with a calculating part for calculating a blood flow velocity associated with the specimen, on the basis of the detected light

According to this aspect, by using that the penetration force of light to a living body depends on wavelength, it is possible to measure the blood flow velocity of each of blood vessels which have different depths from the skin surface. Specifically, by applying light to the surface of a living body, the light penetrating into the body is reflected or scattered by red blood cells flowing in the blood vessel, and its wavelength changes due to the Doppler-shift according to the transfer rate of the red blood cells. On the other hand, as for the light reflected or scattered by skin tissue which can be considered immovable with respect to the red blood cells, the light reaches to the light receiving part without any change in the wavelength. By those lights interfering with each other, an optical beat signal corresponding to the Doppler shift amount is detected on the light receiving part. The calculating part performs an arithmetic process, such as frequency analysis, on the optical beat signal, thereby calculating the velocity of the blood flowing in the blood vessel.

The operation and other advantages of the present invention will become more apparent from the embodiments explained below.

As explained in detail above, according to the light-emitting sensor device of the present invention, it is provided with the substrate, the irradiating part, the light receiving part, and the cap. Thus, it is possible to detect the predetermined type of information, such as a blood flow velocity, on the specimen, highly accurately. Moreover, it is possible to increase the yield and to reduce the manufacturing cost, and it is suitable for mass production.

### BRIEF DESCRIPTION OF DRAWINGS

FIG. 1 is a plan view showing the structure on a sensor part substrate of a sensor part of a blood flow sensor device in a first embodiment.

FIG. 2 is a top view showing the sensor part of the blood flow sensor device in the first embodiment.

FIG. 3 is an A-A′ cross sectional view in FIG. 1.

FIG. 4 is a block diagram showing the structure of the blood flow sensor device in the first embodiment.

FIG. 5 is a conceptual view showing one example of how to use the blood flow sensor device in the first embodiment.

FIG. 6 is a top view showing the sensor part of a blood flow sensor device in a second embodiment.

FIG. 7 is a conceptual view showing that laser lights from three laser diodes in the second embodiment are reflected by a reflective light shielding film formed on corresponding inclined surfaces.

FIG. 8 is a top view showing the sensor part of a blood flow sensor device in a third embodiment.

FIG. 9 is a conceptual view showing that laser light from a laser diode in the third embodiment is reflected by the reflective light shielding film formed on corresponding inclined surface.

FIG. 10 is a cross sectional view having the same concept as in FIG. 3 in a fourth embodiment.

FIG. 11 is a cross sectional view having the same concept as in FIG. 10 in a fifth embodiment.

FIG. 12 is a cross sectional view having the same concept as in FIG. 10 in a modified example.

FIG. 13 is a cross sectional view having the same concept as in FIG. 3 in a sixth embodiment.

### DESCRIPTION OF REFERENCE CODES

100, 102, 103, 104, 105, 106 sensor part
110 sensor part substrate
120, 122, 123 laser diode
130 electrode
150 laser diode drive circuit
160 photodiode
170 photodiode amplifier
200, 202, 203, 204, 206 cap
251 light shielding film
252 reflective light shielding film
290 pinhole
310 A/D converter
320 blood flow velocity DSP
400 embedded resin

### BEST MODE FOR CARRYING OUT THE INVENTION

Hereinafter, embodiments of the present invention will be explained with reference to the drawings. Incidentally, the embodiments below exemplify a blood flow sensor device, which is one example of the light-emitting sensor device of the present invention.

### First Embodiment

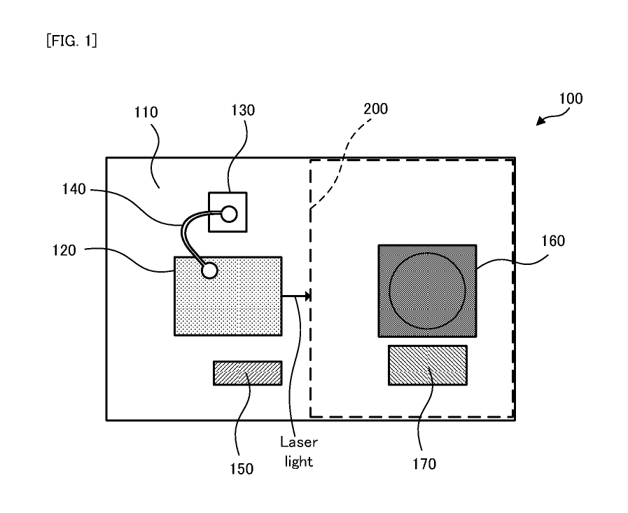

A blood flow sensor device in a first embodiment will be explained with reference to FIG. 1 to FIG. 5.

Firstly, the structure of a sensor part of the blood flow sensor device in the first embodiment will be explained with reference to FIG. 1 to FIG. 3.

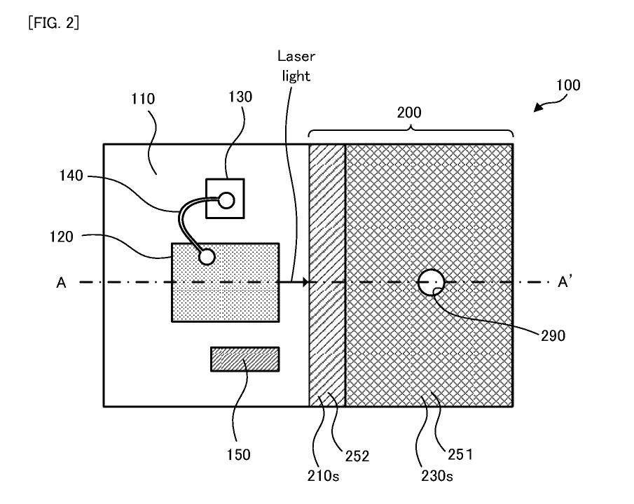

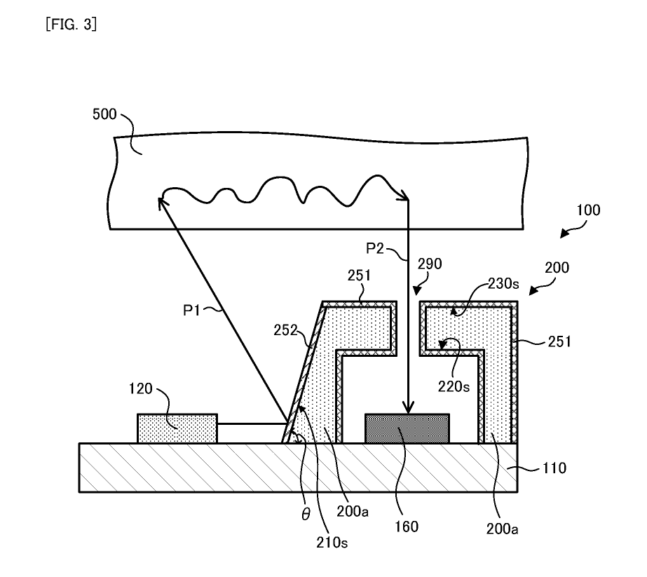

FIG. 1 is a plan view showing the structure on a sensor part substrate of the sensor part of the blood flow sensor device in the first embodiment. FIG. 2 is a top view showing the sensor part of the blood flow sensor device in the first embodiment. FIG. 3 is an A-A′ cross sectional view in FIG. 1. Incidentally, in FIG. 1, for convenience of explanation, a cap 200 shown in FIG. 2 is transparently illustrated as an area surrounded in a dashed line.

As shown in FIG. 1 to FIG. 3, a sensor part 100 of the blood flow sensor device in the first embodiment is provided with a sensor part substrate 110, a laser diode 120, an electrode 130, a wire line 140, a laser diode drive circuit 150, a photodiode 160, a photodiode amplifier 170, and a cap 200.

The sensor part substrate 110 is made of a semiconductor substrate, such as a silicon substrate. On the sensor part substrate 110, the laser diode 120, the laser diode drive circuit 150, the photodiode 160, and the photodiode amplifier 170 are integrated and disposed.

The laser diode 120 is an edge-emitting semiconductor laser, such as an FP laser, and emits laser light to the cap 200 along the substrate surface of the sensor part substrate 100. Incidentally, the laser diode 120 is one example of the “irradiating part” of the present invention. The laser diode 120 is electrically connected to the electrode 130 through the wire line 140. The electrode 130 is electrically connected to an electrode pad (not illustrated) disposed on the bottom of the sensor part substrate 100 by wiring (not illustrate) which penetrates the sensor part substrate 110. Moreover, the other electrode (not illustrate) formed on the bottom surface of the laser diode 120 is electrically connected to an electrode pad (not illustrated) disposed on the bottom of the sensor part substrate 100 by wiring (not illustrate) on the sensor part substrate 110 or wiring (not illustrate) which penetrates the sensor part substrate 110, and it can drive the laser diode 120 by current injection from the exterior of the sensor part 100.

The laser diode drive circuit 150 is a circuit for controlling the drive of the laser diode 120, and it controls the amount of an electric current injected to the laser diode 120.

The photodiode 160 is one example of the “light receiving part” of the present invention, and it functions as a light detector for detecting the light reflected or scattered from a specimen 500 (refer to FIG. 3). Specifically, the photodiode 160 can obtain information about light intensity by converting the light to an electric signal. The photodiode 160 is disposed in parallel with the laser diode 120 on the sensor part substrate 110. The light received on the photodiode 160 is converted to the electric signal and is inputted to the photodiode amplifier 170 via a wire line (not illustrated) and an electrode (not illustrated) formed on the bottom surface of the photodiode 160 or the like.

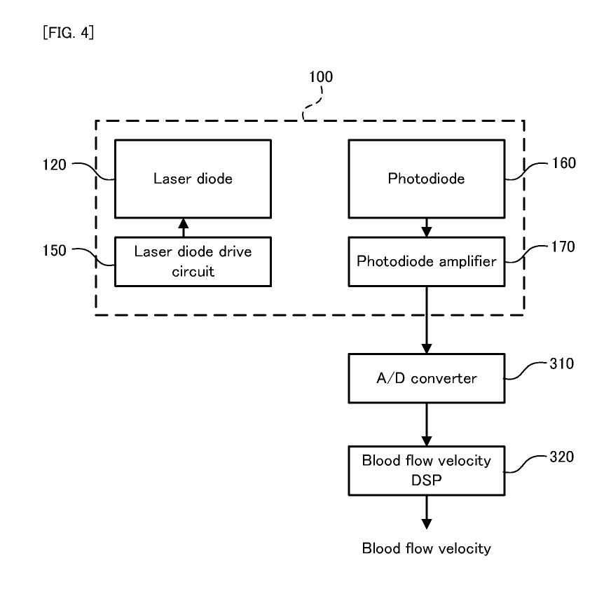

The photodiode amplifier 170 is an amplifier circuit for amplifying the electric signal obtained by the photodiode 160. The photodiode amplifier 170 is electrically connected to the electric pad (not illustrated) disposed on the bottom of the sensor part substrate 100 by the wiring (not illustrate) which penetrates the sensor part substrate 110, and it can output the amplified electric signal to the exterior. The photodiode amplifier 170 is electrically connected to an A/D (Analog to Digital) converter 310 (refer to FIG. 4 described later) disposed in the exterior of the sensor part 100.

The cap 200 has: a cap main body 200a (refer to FIG. 3) for accommodating the photodiode 160; and a light shielding film 251 and a reflective light shielding film 252 formed on the surface of the cap main body 200a.

The cap main body 200a is made of a light shielding resin (e.g. acrylic resin, polycarbonate resin, urea formaldehyde resin, or the like in which light shielding pigments and metal powder are dispersed), and it is formed in a concave shape to accommodate the photodiode 160. The cap main body 200a has an inclined surface 210s, which is inclined at an inclination angle θ (e.g. 60 degrees) to the sensor part substrate 110, as one portion of the outer surface of the cap main body 200a (i.e. a surface which is not opposed to the photodiode 160, out of the surface of the cap main body 200a). In a portion located above the photodiode 160 in the cap main body 200a, a pinhole 290 (refer to FIG. 2 and FIG. 3) is formed which is one example of the “pore” of the present invention. Light P2 from the specimen 500 enters the photodiode 160 via the pinhole 290. The pinhole 290 limits the light entering the photodiode 160. Thus, it is possible to prevent light which does not have to be detected from entering the photodiode 160, thereby increasing detection accuracy. Incidentally, the cap main body 200a may be formed of glass. In this case, the light shielding film 251 as described below is required.

The light shielding film 251 is not necessary if the light shielding resin is used as the material of the cap main body 200a. However, if the cap main body 200a is formed of a material transparent to light, it is made of a metal film having a light shielding property, such as a chromium (Cr) and aluminum (Al), and it is formed on an inner surface 220s of the cap main body 200a (i.e. a surface opposed to the photodiode 160), an outer surface 230s other than the inclined surface 210s out of the outer surface, and the inner surface of the pinhole 290. By virtue of the light shielding film 251, it is possible to prevent unnecessary light from the surroundings of the sensor part 100 from entering the photodiode 160. Incidentally, the diameter of the pinhole 290 is, for example, about 50 μm.

In the pinhole 290, a protective layer may be formed by a resin transparent to the light from the laser diode 120, glass, or the like, or the inside of the pinhole 290 may be filled with the light transparent resin, glass, or the like, in order to improve reliability by preventing the entry of dirt and gas from the exterior.

The reflective light shielding film 252 is made of a metal reflective film (i.e. a film including metal with a high reflective index, such as silver (Ag), aluminum (Al), copper (Cu) and gold (Au)), and it is formed on the inclined surface 210s. The reflective light shielding film 252 reflects the light emitted from the laser diode 120 to go to the specimen 500. By virtue of the reflective light shielding film 252, it is possible to make it certain that the light emitted from the laser diode 120 along the substrate surface of the sensor part substrate 110 enters the specimen 500 disposed to face the substrate surface of the sensor part substrate 110 (i.e. above the sensor part substrate 110 in FIG. 3). Incidentally, an arrow P1 conceptually shows light which is emitted from the laser diode 120, which is reflected by the reflective light shielding film 252, and which is directed to the specimen 500. Moreover, an arrow P2 conceptually shows light which is reflected or scattered by the body tissue of the specimen 500, such as a fingertip, and which enters the sensor part 100 (more specifically, the photodiode 160).

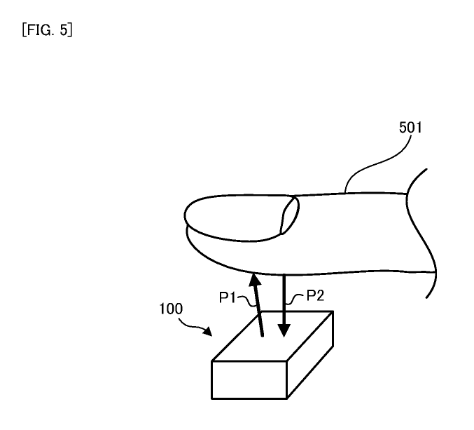

Moreover, the reflective light shielding film 252 also functions as a light shielding device for blocking the direct incidence of the light emitted from the laser diode 120 to the photodiode 160. In other words, the light which is emitted from the laser diode 120 and which goes to the photodiode 160 as it is without being applied to the specimen 500 is blocked by the reflective light shielding film 252. Therefore, it is possible to prevent the light detected by the photodiode 160 from changing due to the light directly going from the laser diode 120 to the photodiode 160. As a result, a blood flow velocity on the specimen 500 can be detected, highly accurately. Incidentally, the measurement of the blood flow velocity will be described later with reference to FIG. 4 and FIG. 5.

In addition, the reflective light shielding film 252 is formed on the inclined surface 210s, which is one portion of the surface of the cap main body 200a made of a resin. Here, particularly in the first embodiment, the cap main body 200a is made of a resin, so that it is easily processed and the inclination angle θ of the inclined surface 210s can be arbitrarily set; namely, the inclination angle θ of the inclined surface 210s can be arbitrarily selected. In other words, the angle of the light from the sensor part 100 (the light from the laser diode 120) entering the specimen 500 can be arbitrarily set.

The cap 200 is bonded to the sensor part substrate 110 by a light shielding adhesive. The light shielding adhesive may be an acrylic, epoxy, polyimide or silicon type adhesive in which conducting particles, such as carbon black, aluminum and silver, are dispersed inside, or an acrylic, epoxy, polyimide or silicon type adhesive in which pigments, such as black pigments, are dispersed inside. Thus, it is reduced by the light shielding adhesive that the unnecessary light from the surroundings of the sensor part 100 passes between the cap 200 and the sensor part substrate 110 and enters the photodiode 160.

The sensor part substrate 110 is desirably a substrate made of a light shielding material; however, it may be formed of a material which can transmit infrared light, such as Si (silicon), in order to unify an electronic circuit and a photodiode. In this case, a light shielding process may be performed separately by using a light shielding resist or the like.

Next, the structure of the entire blood flow sensor device in the first embodiment will be explained with reference to FIG. 4.

FIG. 4 is a block diagram showing the structure of the blood flow sensor device in the first embodiment.

In FIG. 4, the blood flow sensor device in the first embodiment is provided with an A/D converter 310 and a blood flow velocity digital signal processor (DSP) 320, in addition to the aforementioned sensor part 100. Incidentally, in this embodiment, the laser diode drive circuit 150 and the photodiode amplifier 170 are formed on the sensor part substrate 110; however, they may be provided separately from the sensor part 100 without being formed on the sensor part substrate 110 as in the A/D converter 310 and the blood flow velocity DSP 320, or they may be unified on the sensor part substrate 110 including the A/D converter 310 and the blood flow velocity DSP 320. Alternatively, other substrates having their respective functions may be laminated with the sensor part substrate 110, and they may be mounted in an electrically connecting method or the like by wiring and through-hole interconnection. By bringing the A/D converter 310 and the blood flow velocity DSP 320 close to the sensor part substrate 110, a sufficient SN ratio (Signal to Noise Ratio) and a sufficient band can be ensured in weak or faint signal processing.

The A/D converter 310 converts the electric signal outputted from the photodiode amplifier 170, from an analog signal to a digital signal. In other words, the electric signal obtained by the photodiode 160 is amplified by the photodiode amplifier 170, and then it is converted to the digital signal by the A/D converter 310. The A/D converter 310 outputs the digital signal to the blood flow velocity DSP 320.

The blood flow velocity DSP 320 is one example of the “calculating part” of the present invention, and it calculates the blood flow velocity by performing a predetermined arithmetic process on the digital signal inputted from the A/D converter 310.

Next, the measurement of the blood flow velocity by the blood flow sensor device in the first embodiment will be explained with reference to FIG. 5 in addition to FIG. 4.

FIG. 5 is a conceptual view showing one example of how to use the blood flow sensor device in the first embodiment.

As shown in FIG. 5, the blood flow sensor device in the first embodiment measures the blood flow velocity by irradiating a fingertip 501, which is one example of the specimen 500 (refer to FIG. 3), with laser light with a predetermined wavelength (e.g. shortwave light with a wavelength of 780 nm, or long-wave light with a wavelength of 830 nm) by using the laser diode 120. At this time, a portion irradiated with the laser light is more desirably a portion in which blood capillaries are distributed densely in a position relatively close to the epidermis (e.g. hand, leg, face, ear, or the like).

In FIG. 5, the laser light applied to the fingertip 501 penetrates to depth according to its wavelength, and it is reflected or scattered by the body tissue of the fingertip 501, such as blood flowing in blood vessels like the blood capillaries or the like and skin cells which constitute the epidermis. Incidentally, in FIG. 5, an arrow P1 conceptually shows the light going to the fingertip 501 from the sensor part 100. Moreover, an arrow P2 conceptually shows the light entering the sensor part 100 after being reflected or scattered by the body tissue of the fingertip 501. Then, the Doppler shift occurs in the light reflected or scattered by red blood cells flowing in the blood vessels, and the wavelength of the light changes depending on the transfer rate of the red blood cells or the rate at which the blood flows (i.e. the blood flowing velocity). On the other hand, as for the light reflected or scattered by the skin cells or the like which can be considered immovable with respect to the red blood cells, the wavelength of the light does not change. By those lights interfering with each other, an optical beat signal corresponding to the Doppler shift amount is detected on the photodiode 160 (refer to FIG. 4). The blood flow velocity DSP 320 (refer to FIG. 4) performs frequency analysis on the optical beat signal detected by the photodiode 160 and calculates the Doppler shift amount, thereby calculating the blood flow velocity.

Back in FIG. 1 to FIG. 3 again, particularly in the embodiment, there is provided the cap 200, which has: the cap main body 200a made of a resin; and the reflective light shielding film 252 formed on the inclined surface 210s of the cap main body 200a, as described above. Thus, it is possible to make it certain that the light emitted from the laser diode 120 along the substrate surface of the sensor part substrate 110 enters the specimen 500 by being reflected by the reflective light shielding film 252. Moreover, by the reflective light shielding film 252, it is possible to prevent the light emitted from the laser diode 120 along the substrate surface of the sensor part substrate 110, from entering the photodiode 160 as it is without being applied to the specimen 500. Thus, it is possible to prevent the light detected by the photodiode 160 from changing due to the light directly going to the photodiode 160 from the laser diode 120.

Moreover, the cap 200 is formed of the cap main body 200a made of a resin; and the light shielding film 251 and the reflective light shielding film 252 formed on the surface of the cap main body 200a, so that it is easily processed and each process in a manufacturing process can be simplified or reduced. By this, it is possible to increase a yield and to reduce manufacturing cost. Thus, the blood flow sensor device in the first embodiment is suitable for mass production.

### Second Embodiment

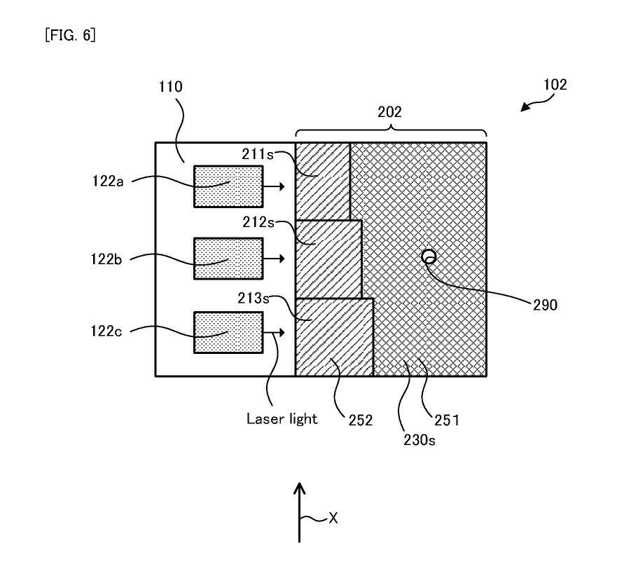

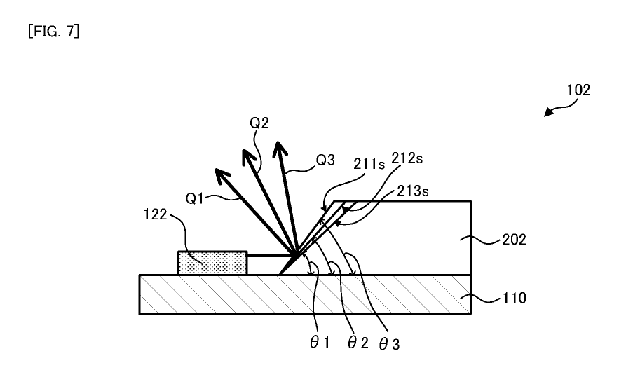

A blood flow sensor device in a second embodiment will be explained with reference to FIG. 6 and FIG. 7.

FIG. 6 is a top view showing the sensor part of the blood flow sensor device in the second embodiment. FIG. 7 is a conceptual view showing that laser lights from three laser diodes in the second embodiment are reflected by the reflective light shielding film formed on corresponding inclined surfaces. Incidentally, FIG. 7 shows the sensor part 100 in accordance with the side surface of the sensor part 100 viewed in an X direction (i.e. in an upward direction) in FIG. 6. Incidentally, in FIG. 6 and FIG. 7, the same constituents as those in the first embodiment shown in FIG. 1 to FIG. 5 will carry the same reference numerals, and the explanation thereof will be omitted, as occasion demands.

The blood flow sensor device in the second embodiment is different from the blood flow sensor apparatus in the first embodiment described above in the point that it is provided with a sensor part 102 instead of the sensor part 100 in the first embodiment described above, and it is constructed in substantially the same manner as the blood flow sensor apparatus in the first embodiment described above in other points.

In FIG. 6 and FIG. 7, the sensor part 102 of the blood flow sensor apparatus in the second embodiment is different from the sensor part 100 of the blood flow sensor apparatus in the first embodiment described above in the point that it is provided with three laser diodes 122 (i.e. laser diodes 122a, 122b, and 122c) instead of the laser diode 120 in the first embodiment described above and in the point that it is provided with a cap 202 instead of the cap 200 in the first embodiment described above, and it is constructed in substantially the same manner as the sensor part 100 of the blood flow sensor apparatus in the first embodiment described above in other points.

Incidentally, in FIG. 6, as for a laser diode drive circuit, an electrode, and a wire line for driving the three laser diodes 122, the illustration will be omitted. The laser diode drive circuit, the electrode, and the wire line may be disposed on the sensor part substrate 110 in substantially the same manner as the aforementioned first embodiment, or they may be disposed separately from the sensor part 102 without being formed on the sensor part substrate 110.

In FIG. 6 and FIG. 7, particularly in the second embodiment, the three laser diodes 122a, 122b, and 122c are disposed on the sensor part substrate 110. At the same time, inclined surfaces 211s, 212s, and 213s are formed on the cap 202, which are inclined to the substrate surface of the sensor part substrate 110 at mutually different inclination angles in accordance with the respective laser diodes 122.

The laser diodes 122a, 122b, and 122c are edge-emitting semiconductor lasers and emit laser lights to the cap 202. More specifically, the laser diode 122a emits laser light along the substrate surface of the sensor part substrate 110, to the inclined surface 211s formed on the cap 202. The laser diode 122b emits laser light along the substrate surface of the sensor part substrate 110, to the inclined surface 212s formed on the cap 202. The laser diode 122c emits laser light along the substrate surface of the sensor part substrate 110, to the inclined surface 213s formed on the cap 202.

The cap 202 is different from the cap 200 in the first embodiment described above in the point that it has the three inclined surfaces 211s, 212s, and 213s instead of the inclined surface 210s in the first embodiment described above, and it is constructed in substantially the same manner as the cap 200 in the first embodiment described above in other points.

The inclined surfaces 211s, 212s, and 213s are inclined to the substrate surface of the sensor part substrate 110 at the mutually different inclination angles; namely, an inclination angle θ1 at which the inclined surface 211s is inclined to the substrate surface of the sensor part substrate 110, an inclination angle θ2 at which the inclined surface 212s is inclined to the substrate surface of the sensor part substrate 110, and an inclination angle θ3 at which the inclined surface 213s is inclined to the substrate surface of the sensor part substrate 110 are different from each other. On the inclined surfaces 211s, 212s, and 213s, the reflective light shielding film 252 is formed which is made of a metal reflective film.

Thus, the lights emitted from the three laser diodes 122a, 122b, and 122c can be reflected to mutually different portions on the specimen, by the reflective light shielding film 252 formed on the three inclined surfaces 211s, 212s, and 213s, which are inclined at the mutually different inclination angles. Incidentally, in FIG. 7, an arrow Q1 conceptually shows the light which is emitted from the laser diode 122a, which is reflected by a portion formed on the inclined surface 211s of the reflective light shielding film 252, and which goes to the specimen. An arrow Q2 conceptually shows the light which is emitted from the laser diode 122b, which is reflected by a portion formed on the inclined surface 212s of the reflective light shielding film 252, and which goes to the specimen. An arrow Q3 conceptually shows the light which is emitted from the laser diode 122c, which is reflected by a portion formed on the inclined surface 213s of the reflective light shielding film 252, and which goes to the specimen.

Therefore, the blood flow velocity in the mutually different three portions on the specimen can be detected, more quickly. In other words, the blood flow velocity in the three portions on the specimen can be detected without changing a relative position relation between the specimen and the sensor part 102.

Incidentally, in the measurement of the blood flow velocity, the three laser diodes 122a, 122b, and 122c sequentially emit the laser lights, and the photodiode 160 detects the light from the specimen in a time-sharing manner for each of the laser diodes 122a, 122b, and 122c.

Incidentally, the three laser diodes 122a, 122b, and 122c may be semiconductor lasers which emit respective laser lights with the same wavelength, or semiconductor lasers which emit respective laser lights with mutually different wavelengths. Here, if the three laser diodes 122a, 122b, and 122c are formed from the semiconductor lasers each of which emits respective one of the laser lights with mutually different wavelengths, the measurement in various depths of the specimen can be performed.

### Third Embodiment

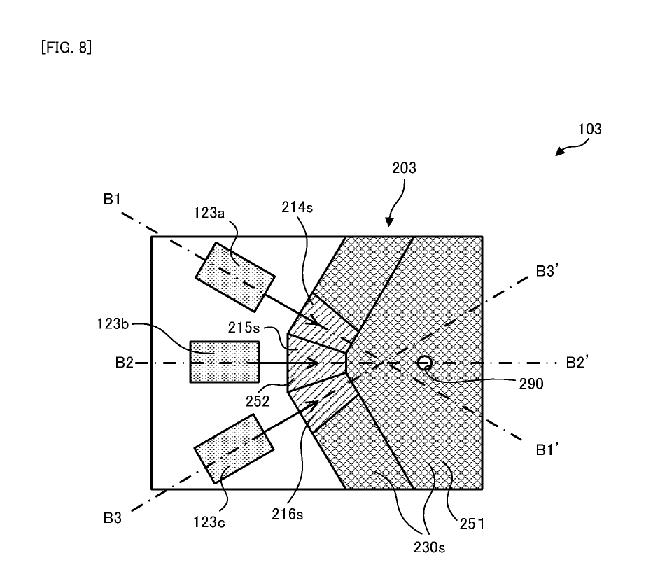

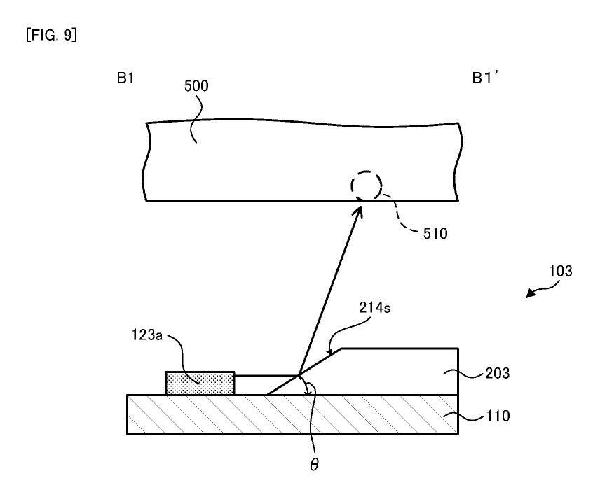

A blood flow sensor device in a third embodiment will be explained with reference to FIG. 8 and FIG. 9.

FIG. 8 is a top view showing the sensor part of the blood flow sensor device in the third embodiment. FIG. 9 is a conceptual view showing that laser light from a laser diode in the third embodiment is reflected by a reflective light shielding film formed on corresponding inclined surface. Incidentally, FIG. 9 schematically shows the light reflected by the light shielding film in accordance with a cross section in a case where a sensor part 103 is cut along a B1-B1′ line in FIG. 8. A case where the sensor part 103 is cut along a B2-B2′ line in FIG. 8 and a case where the sensor part 103 is cut along a B3-B3′ line in FIG. 8 are also substantially the same as in FIG. 9. Incidentally, in FIG. 8 and FIG. 9, the same constituents as those in the first embodiment shown in FIG. 1 to FIG. 5 will carry the same reference numerals, and the explanation thereof will be omitted, as occasion demands.

The blood flow sensor device in the third embodiment is different from the blood flow sensor apparatus in the first embodiment described above in the point that it is provided with the sensor part 103 instead of the sensor part 100 in the first embodiment described above, and it is constructed in substantially the same manner as the blood flow sensor apparatus in the first embodiment described above in other points.

In FIG. 8 and FIG. 9, the sensor part 103 of the blood flow sensor apparatus in the third embodiment is different from the sensor part 100 of the blood flow sensor apparatus in the first embodiment described above in the point that it is provided with three laser diodes 123 (i.e. laser diodes 123a, 123b, and 123c) instead of the laser diode 120 in the first embodiment described above and in the point that it is provided with a cap 203 instead of the cap 200 in the first embodiment described above, and it is constructed in substantially the same manner as the sensor part 100 of the blood flow sensor apparatus in the first embodiment described above in other points.

Incidentally, in FIG. 8, as for a laser diode drive circuit, an electrode, and a wire line for driving the three laser diodes 123, the illustration will be omitted. The laser diode drive circuit, the electrode, and the wire line may be disposed on the sensor part substrate 110 in substantially the same manner as the aforementioned first embodiment, or they may be disposed separately from the sensor part 103 without being formed on the sensor part substrate 110.

In FIG. 8 and FIG. 9, particularly in the third embodiment, the three laser diodes 123a, 123b, and 123c are disposed on the sensor part substrate 110. At the same time, inclined surfaces 214s, 215s, and 216s are formed on the cap 203, which are inclined to the substrate surface of the sensor part substrate 110, in accordance with the respective laser diodes 123. On the inclined surfaces 214s, 215s, and 216s, the reflective light shielding film 252 is formed which is made of a metal reflective film.

The laser diodes 123a, 123b, and 123c are edge-emitting semiconductor lasers and emit laser lights with mutually different wavelengths to the cap 203. More specifically, the laser diode 123a emits laser light along the substrate surface of the sensor part substrate 110, to the inclined surface 214s formed on the cap 203. The laser diode 123b emits laser light along the substrate surface of the sensor part substrate 110, to the inclined surface 215s formed on the cap 203. The laser diode 123c emits laser light along the substrate surface of the sensor part substrate 110, to the inclined surface 216s formed on the cap 203.

The cap 203 is different from the cap 200 in the first embodiment described above in the point that it has the three inclined surfaces 214s, 215s, and 216s instead of the inclined surface 210s in the first embodiment described above, and it is constructed in substantially the same manner as the cap 200 in the first embodiment described above in other points.

In the third embodiment, in particular, the inclined surfaces 214s, 215s, and 216s are disposed such that the reflected lights on the inclined surfaces of the laser lights from the laser diodes 123a, 123b, and 123c are applied to the same portion on the specimen.

In other words, the orientations and inclination angles θ of the inclined surfaces 214s, 215s, and 216s are adjusted in accordance with the layout of the laser diodes 123a, 123b, and 123c such that each of light obtained by that the light emitted from the laser diode 123a is reflected by a portion formed on the inclined surface 214s of the reflective light shielding film 252, light obtained by that the light emitted from the laser diode 123b is reflected by a portion formed on the inclined surface 215s of the reflective light shielding film 252, and light obtained by that the light emitted from the laser diode 123c is reflected by a portion formed on the inclined surface 216s of the reflective light shielding film 252 enters one portion 510 on the specimen 500.

Thus, it is possible to detect the blood flow velocity by applying the laser lights with mutually different wavelengths to the same portion on the specimen (e.g. the portion 510 in FIG. 9).

Incidentally, in the measurement of the blood flow velocity, the three laser diodes 123a, 123b, and 123c sequentially emit the laser lights, and the photodiode 160 detects the light from the specimen in a time-sharing manner for each of the laser diodes 123a, 123b, and 123c.

### Fourth Embodiment

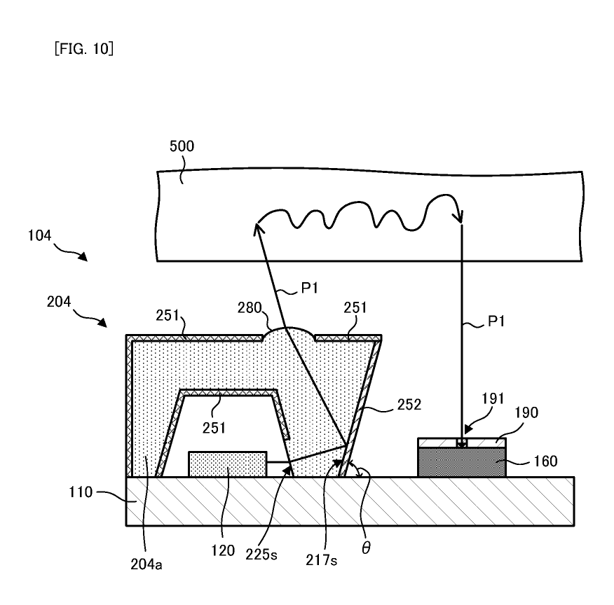

A blood flow sensor device in a fourth embodiment will be explained with reference to FIG. 10.

FIG. 10 is a cross sectional view having the same concept as in FIG. 3 in the fourth embodiment. Incidentally, in FIG. 10, the same constituents as those in the first embodiment shown in FIG. 1 to FIG. 5 will carry the same reference numerals, and the explanation thereof will be omitted, as occasion demands.

The blood flow sensor device in the fourth embodiment is different from the blood flow sensor apparatus in the first embodiment described above in the point that it is provided with the sensor part 104 instead of the sensor part 100 in the first embodiment described above, and it is constructed in substantially the same manner as the blood flow sensor apparatus in the first embodiment described above in other points.

In FIG. 10, the sensor part 104 of the blood flow sensor apparatus in the fourth embodiment is different from the sensor part 100 of the blood flow sensor apparatus in the first embodiment described above in the point that it is provided with a cap 204 including a material which transmits the light from the laser diode 120 instead of the cap 200 in the first embodiment described above and in the point that it is further provided with a light shielding film 190, which is one example of the “light receiving part upper surface light shielding film” of the present invention, and it is constructed in substantially the same manner as the sensor part 100 of the blood flow sensor apparatus in the first embodiment described above in other points.

In FIG. 10, the cap 204 is made of a cap main body 204a for accommodating the laser diode 120; and a light shielding film 251 an a reflective light shielding film 252 formed on the surface of the cap main body 204a.

The cap main body 204a is made of a transparent resin (e.g. acrylic resin), and it is formed in a concave shape to accommodate the laser diode 120. The cap main body 204a has an inclined surface 217s, which is inclined at an inclination angle θ (e.g. 60 degrees) to the sensor part substrate 110, as one portion of the outer surface of the cap main body 204a (i.e. a surface which is not opposed to the laser diode 120, out of the surface of the cap main body 204a). On the inclined surface 217s, the reflective light shielding film 252 made of a metal reflective film is formed. Moreover, as one portion of the cap main body 204a, a lens 280 is formed on the upper surface side of the cap main body 204a. The lens 280 can be molded simultaneously with the cap main body 204a. The lens 280 can collimate the laser light from the laser diode 120 (in other words, the light emitted from the laser diode 120 and reflected by the reflective light shielding film 252). In other words, the lens 280 can change the laser light entering the specimen 500 to parallel light and increase the intensity and usability of the laser light.

The light shielding film 251 is formed on a surface other than a refracting surface 225s described later out of the inner surface of the cap main body 204a (i.e. a surface opposed to the photodiode 160) and a surface other than an area where the inclined surface 217s and the lens 280 are formed out of the outer surface of the cap main body 204a.

The refracting surface 225s constitutes one portion of the inner surface of the cap main body 204a and refracts the laser light emitted from the laser diode 120 to go to the reflective light shielding film 252 formed on the inclined surface 217s.

In the fourth embodiment, in particular, it is provided with the cap 204 as constructed above, so that the light emitted from the laser diode 120 is refracted by the refracting surface 225s, is transmitted through the inside of the cap main body 204a, and then is reflected by the reflective light shielding film 252 formed on the inclined surface 217s, which is one portion of the outer surface of the cap main body 204a, to go to the specimen 500. Then, the reflected light is collimated by the lens 280 and is applied to the specimen 500. Thus, for example, by changing the inclination angle of each of the refracting surface 225s and the inclined surface 217s to the substrate surface, it is possible to change the path of the light emitted from the laser diode 120 to the specimen 500. In other words, in designing the path of the light emitted from the laser diode 120 to the specimen 500, the inclination angles of the inclined surface 217s and the refracting surface 225s can be set as design parameters.

In FIG. 10, the light shielding film 190 is made of a light shielding resin in a film shape and is formed to cover the upper surface of the photodiode 160. The light shielding film 190 has a pinhole 191 formed. The light from the specimen 500 enters the photodiode 160 via the pinhole 191. The pinhole 191 limits the light entering the photodiode 160. Thus, it is possible to prevent the light which does not have to be detected from entering the photodiode 160, thereby increasing the detection accuracy. Incidentally, in the pinhole 191, a protective layer may be formed by a resin transparent to the light from the laser diode 120, glass, or the like, or the inside of the pinhole 191 may be filled with the light transparent resin, glass, or the like, in order to improve reliability by preventing the entry of dirt and gas from the exterior.

### Fifth Embodiment

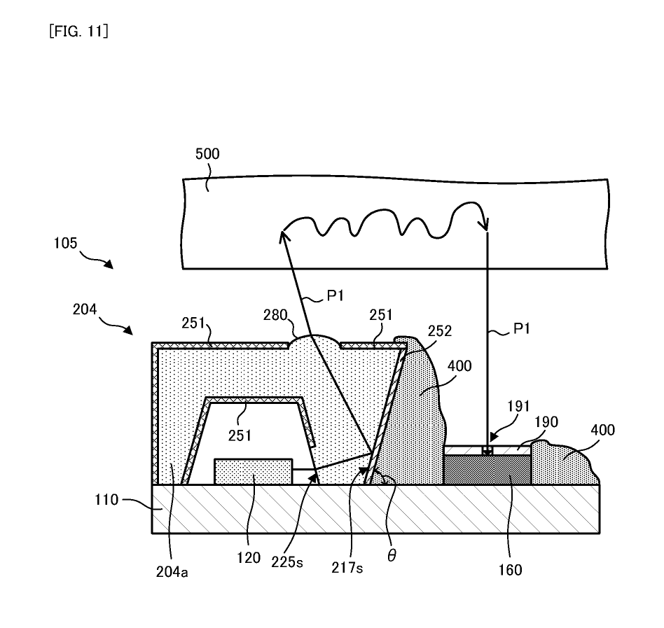

A blood flow sensor device in a fifth embodiment will be explained with reference to FIG. 11.

FIG. 11 is a cross sectional view having the same concept as in FIG. 10 in the fifth embodiment. Incidentally, in FIG. 11, the same constituents as those in the fourth embodiment shown in FIG. 10 will carry the same reference numerals, and the explanation thereof will be omitted, as occasion demands.

The blood flow sensor device in the fifth embodiment is different from the blood flow sensor apparatus in the fourth embodiment described above in the point that it is provided with the sensor part 105 instead of the sensor part 104 in the fourth embodiment described above, and it is constructed in substantially the same manner as the blood flow sensor apparatus in the fourth embodiment described above in other points.

In FIG. 11, the sensor part 105 of the blood flow sensor apparatus in the fifth embodiment is different from the sensor part 104 of the blood flow sensor apparatus in the fourth embodiment described above in the point that it is further provided with an embedded resin 400, which is one example of the “resin part” of the present invention, and it is constructed in substantially the same manner as the sensor part 104 of the blood flow sensor apparatus in the fourth embodiment described above in other points.

In FIG. 11, the embedded resin 400 is made of a light shielding resin and is formed to cover the reflective light shielding film 252 and to surround the photodiode 160 viewed in a two-dimensional manner on the sensor part substrate 110. The embedded resin 410 can prevent the oxidation of the reflective light shielding film 252 made of a metal reflective film such as an Ag film and an Al film, and allows it to be reduced that the unnecessary light from the surroundings of the photodiode 160 enters the photodiode 160. Therefore, the durability or reliability of the sensor part 105 can be increased, and the detection accuracy can be also increased.

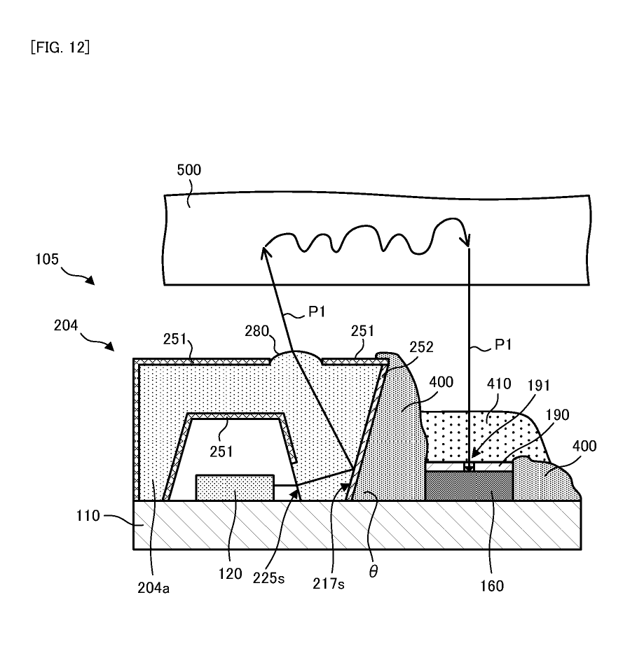

FIG. 12 is a cross sectional view having the same concept as in FIG. 10 in a modified example.

As shown as the modified example in FIG. 12, the sensor part 106 may be mounted on another structure (not illustrated) before the upper portion of the light shielding film 190 is molded (or shaped) to wrap it with the resin 410 transparent to the light from the laser diode 120. By virtue of such construction, it is possible to stably hold the sensor part 105 after being mounted on another structure, thereby significantly increasing the reliability such as a performance to environment. Incidentally, the transparent resin part 410 may be molded to wrap the entire sensor part 105. Even in this case, it is possible to stably hold the sensor part 105 after being mounted on another structure, thereby significantly increasing the reliability such as a performance to environment.

### Sixth Embodiment

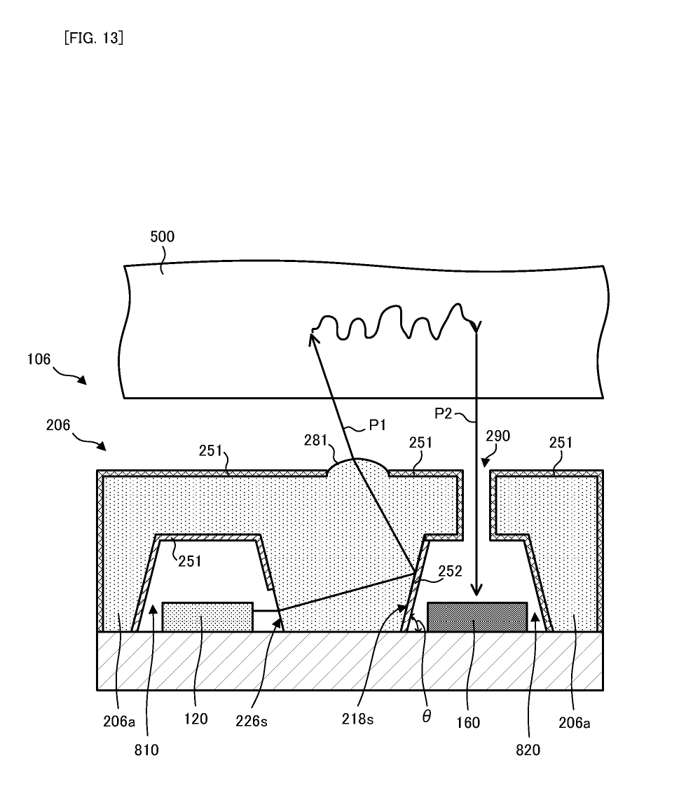

A blood flow sensor device in a sixth embodiment will be explained with reference to FIG. 13.

FIG. 13 is a cross sectional view having the same concept as in FIG. 3 in the sixth embodiment. Incidentally, in FIG. 13, the same constituents as those in the first embodiment shown in FIG. 1 to FIG. 5 will carry the same reference numerals, and the explanation thereof will be omitted, as occasion demands.

In FIG. 13, a sensor part 106 of the blood flow sensor apparatus in the sixth embodiment is different from the sensor part 100 of the blood flow sensor apparatus in the first embodiment described above in the point that it is provided with a cap 206 instead of the cap 200 in the first embodiment described above, and it is constructed in substantially the same manner as the sensor part 100 of the blood flow sensor apparatus in the first embodiment described above in other points.

In FIG. 13, the cap 206 is made of a cap main body 206a for accommodating the laser diode 120 and the photodiode 160; and a light shielding film 251 an a reflective light shielding film 252 formed on the surface of the cap main body 206a.

The cap main body 206a is made of a transparent resin (e.g. acrylic resin), and it has two concave portions 810 and 820 which can separately accommodate the laser diode 120 and the photodiode 160, respectively. The laser diode 120 is accommodated in the concave portion 810 of the cap main body 206a, and the photodiode 160 is accommodated in the concave portion 820 of the cap main body 206a.

The cap main body 206a has an inclined surface 218s, which is inclined at an inclination angle θ (e.g. 60 degrees) to the sensor part substrate 110, as one portion of the inner surface of the concave portion 820 (i.e. a surface opposed to the photodiode 160, out of the surface of the concave portion 820). On the inclined surface 218s, the reflective light shielding film 252 made of a metal reflective film is formed. Moreover, the cap main body 206a has a refracting surface 226s which refracts the laser light emitted from the laser light to go to the inclined surface 218s, as one portion of the inner surface of the concave portion 810 (i.e. a surface opposed to the laser diode 120, out of the surface of the concave portion 810).

As one portion of the cap main body 206a, a lens 281 is formed on the upper surface side of the cap main body 206a. The lens 281 can be molded simultaneously with the cap main body 206a. The lens 281 can collimate the laser light from the laser diode 120 (in other words, the light emitted from the laser diode 120 and reflected by the reflective light shielding film 252). In other words, the lens 280 can change the laser light entering the specimen 500 to parallel light and increase the intensity and usability of the laser light. In a portion located above the photodiode 160 in the cap main body 206a, a pinhole 290 is formed. The light from the specimen 500 enters the photodiode 160 via the pinhole 290.

The light shielding film 251 is formed on a surface other than the refracting surface 226s and the inclined surface 217 out of the inner surface of the cap main body 206a (i.e. the inner surfaces of the concave portions 810 and 820, in other words, the surfaces opposed to the laser diode 120 and the photodiode 160) and a surface other than an area where the lens 281 is formed out of the outer surface of the cap main body 206a (i.e. surfaces which are not opposed to the laser diode 120 and the photodiode 160).

In the sixth embodiment, in particular, it is provided with the cap 206 as constructed above, so that the light emitted from the laser diode 120 is refracted by the refracting surface 226s, is transmitted through the inside of the cap main body 206a, and then is reflected by the reflective light shielding film 252 formed on the inclined surface 218s, which is one portion of the inner surface of the concave portion 820 of the cap main body 206a, to go to the specimen 500. Then, the reflected light is collimated by the lens 281 and is applied to the specimen 500. Thus, for example, by changing the inclination angle of each of the refracting surface 226s and the inclined surface 218s to the substrate surface, it is possible to change the path of the light emitted from the laser diode 120 to the specimen 500. In other words, in designing the path of the light emitted from the laser diode 120 to the specimen 500, the inclination angles of the inclined surface 218s and the refracting surface 226s can be set as design parameters.

Moreover, particularly in this embodiment, the cap 206 is formed such that the laser diode 120 and the photodiode 160 are accommodated in the two concave portions 810 and 820, respectively, so that the laser diode 120 and the photodiode 160 can be protected by the cap 206. Thus, the durability of reliability of the sensor part 106 can be increased.

In addition, the sensor part 106 in FIG. 13 may be mounted on another structure (not illustrated) before the upper portion of the pinhole 290 or the entire sensor part 106 is molded to wrap it with a resin transparent to the light from the laser diode 120. By virtue of such construction, it is possible to stably hold the sensor part 106 after being mounted on another structure, thereby significantly increasing the reliability such as a performance to environment.

The present invention is not limited to the aforementioned example, but various changes may be made, if desired, without departing from the essence or spirit of the invention which can be read from the claims and the entire specification. A light-emitting sensor device, which involves such changes, is also intended to be within the technical scope of the present invention.

### INDUSTRIAL APPLICABILITY

The light-emitting sensor device of the present invention can be applied to a blood flow sensor device or the like capable of measuring a blood flow velocity or the like.

1. A light-emitting sensor device comprising:
a substrate;
an irradiating part, disposed on said substrate, for applying light to a specimen;
a light receiving part, disposed on said substrate, for detecting light from the specimen caused by the applied light; and
a cap, disposed on said substrate, which has (i) a cap main body for accommodating at least one of said irradiating part and said light receiving part and (ii) a reflective light shielding film which is one portion of a surface of the cap main body, which is formed on an inclined surface inclined to a substrate surface of said substrate, which reflects the light emitted from said irradiating part to go to the specimen, and which blocks incidence of the light emitted from said irradiating part to said light receiving part.

2. The light-emitting sensor device according to claim 1, wherein the cap main body is formed of a resin, and a light shielding film is formed at least partially on a surface other than the inclined surface out of a surface of the cap main body.

3. The light-emitting sensor device according to claim 1, wherein the cap main body accommodates said light receiving part as the at least one and has a pore for transmitting light from the specimen.

4. The light-emitting sensor device according to claim 1, wherein
said irradiating part has a plurality of light sources, and
the cap main body has a plurality of inclined surfaces, each of which is formed in accordance with respective one of a plurality of lights emitted from the plurality of light sources and which are inclined to the substrate surface at mutually different angles.

5. The light-emitting sensor device according to claim 4, wherein the plurality of light sources are a plurality of semiconductor lasers, each of which emits respective one of laser lights with mutually different wavelengths.

6. The light-emitting sensor device according to claim 5, wherein the plurality of inclined surfaces are arranged such that a plurality of reflected lights, obtained by reflecting the plurality of lights with the reflective light shielding film, are applied to a same portion on the specimen.

7. The light-emitting sensor device according to claim 1, wherein
the cap main body accommodates said irradiating part as the at least one and is made of a transparent member which can transmit the light emitted from said irradiating part,
the inclined surface is one portion of an outer surface located on a side which is not opposed to said irradiating part, out of a surface of the cap main body, and
the cap main body has a refracting surface which refracts the light emitted from said irradiating part to go to the reflective light shielding film.

8. The light-emitting sensor device according to claim 1, wherein
the cap main body accommodates said irradiating part as the at least one and is made of a transparent member which can transmit the light emitted from said irradiating part,
the inclined surface is one portion of an outer surface located on a side which is not opposed to said irradiating part, out of a surface of the cap main body, and
said light-emitting sensor device further comprises a resin part formed of a light shielding resin to cover the reflective light shielding film and to surround said light receiving part.

9. The light-emitting sensor device according to claim 1, further comprising a light receiving part upper surface light shielding film, which is disposed on an upper surface of said light receiving part, which is made of a light shielding material, and which is to transmit light from the specimen.

10. The light-emitting sensor device according to claim 1, wherein
the cap main body accommodates said irradiating part and said light receiving part and is made of a transparent member which can transmit the light emitted from said irradiating part,
the inclined surface is one portion of a light-receiving-part-side inner surface opposed to said light receiving part, out of a surface of the cap main body, and
one portion of an irradiating-part-side inner surface opposed to said irradiating part out of the surface of the cap main body is formed as a refracting surface which refracts the light emitted from said irradiating part to go to the reflective light shielding film.

11. The light-emitting sensor device according to claim 1, wherein said irradiating part has an edge-emitting semiconductor laser for emitting laser light along the substrate surface as the light.

12. The light-emitting sensor device according to claim 1, further comprising a calculating part for calculating a blood flow velocity associated with the specimen, on the basis of the detected light
